(19) United States
(12) Patent Application Publication
(10) Pub. No.: US 2021/0074387 A1
(43) Pub. Date: Mar. 11, 2021

(54) SIMULATION METHOD FOR ANALYZING DIFFUSION PROPERTY OF WATER-SOLUBLE MONOMER IN HYDROGEL MEMBRANE

(71) Applicant: Energy Research Institute of Jiangxi Academy of Sciences, Nanchang (CN)

(72) Inventors: Zhaohuan MAI, Nanchang (CN); Jihai XIONG, Nanchang (CN); Cheng JIANG, Nanchang (CN); Jiujiu WU, Nanchang (CN); Shuanglin GUI, Nanchang (CN); Bing YAN, Nanchang (CN); Qizhen YI, Nanchang (CN)

(21) Appl. No.: 16/828,095

(22) Filed: Mar. 24, 2020

(30) Foreign Application Priority Data
Sep. 11, 2019 (CN) 201910859102.1

(51) Int. Cl. G16C 10/00; G16C 60/00

(52) U.S. Cl. CPC G16C 10/00; G16C 60/00

(57) ABSTRACT

The present invention belongs to the field of environmental materials, and discloses a simulation method for analyzing a diffusion property of a water-soluble monomer in a hydrogel membrane. The method specifically includes the following steps: 1) selecting a hydrogel membrane material and a water-soluble monomer to be simulated; 2) constructing an initial model; 3) optimizing a molecular dynamics model of the hydrogel membrane system; 4) performing a molecular dynamics simulation of the optimized model; 5) drawing a mean square displacement-time (MSD-t) curve; and 6) calculating a diffusion coefficient of a water-soluble monomer molecule in the hydrogel membrane system. The present invention analyzes the diffusion performance of the water-soluble monomer molecule in the hydrogel membrane system on a molecular level. The present invention quantitatively calculates a diffusion coefficient of the water-soluble monomer in the hydrogel membrane system and further obtains an influence of a hydrogel on interfacial polymerization.

### TECHNICAL FIELD

The present invention belongs to the field of environmental materials, and in particular, relates to a simulation method for analyzing a diffusion property of a water-soluble monomer in a hydrogel membrane for the preparation of a high-performance membrane material.

### BACKGROUND

With the shortage of water resources and the increasingly serious pollution of water, membrane separation technology, as one of the economic and efficient technologies for sewage treatment, seawater desalination and brackish water desalination, has broad market applications. Membrane materials, as the core of membrane separation technology, directly affect the separation performance of membranes and the application of membrane technology. The preparation of high-performance membrane materials is a hotspot for continuous development and research in the industrial and academic areas. At present, commercial reverse osmosis (RO) membranes, nanofiltration (NF) membranes and organic solvent-tolerant NF composite membranes are generally prepared through the interfacial polymerization of an amine monomer in an aqueous phase and a polyacyl chloride monomer in an organic phase. The water-soluble monomer and the polyacyl chloride monomer form a selective layer (polyamide, PA) on the surface of a substrate. In the process of interfacial polymerization, the concentration of the monomers, the reaction time and the structure of the substrate are the key factors affecting the performance of the finally prepared polyamide thin-film composite (PA-TFC) membrane.

Recently, researchers have added polymers (such as Kevlar fiber), instead of a conventional ultrafiltration (UF) membrane as a substrate into a reaction solution to prepare a superior ultra-thin PA-TFC membrane. Great progress has been made in the synthesis and modification of membrane materials. However, there is still insufficient research and explanation on the microstructural properties and mechanism of interfacial polymerization for the preparation of high-performance membrane materials, making the preparation process blind. Therefore, it is important to research the microstructure characteristics and mechanism of interfacial polymerization for the preparation of high-performance composite membranes. At present, widely used experimental characterization and detection methods include scanning electron microscope (SEM), transmission electron microscope (TEM) and atomic force microscope (AFM). They are difficult to meet requirements for the quantitative analysis of surface microscopic characteristics and dynamic changes of water-soluble monomers in hydrogel membrane systems and in interfacial polymerization at an atomic or molecular level. It is also difficult to explain the interfacial polymerization mechanism at the atomic or molecular level.

### SUMMARY

To solve the above problems, the present invention provides a simulation method for analyzing a diffusion property of a water-soluble monomer in a hydrogel membrane. The present invention calculates a diffusion coefficient of a water-soluble monomer in a hydrogel membrane system by a method of molecular dynamics simulation. The present invention provides a theoretical basis for exploring an influence of a gel system on interfacial polymerization and membrane separation performance, and provides data support for preparing a high-performance membrane material by using a hydrogel.

The present invention has the following technical solutions.

A simulation method for analyzing a diffusion property of a water-soluble monomer in a hydrogel membrane includes the following steps.

1) selecting a hydrogel membrane material and a water-soluble monomer to be simulated;

2) constructing an initial model constructing a molecular dynamics model of a hydrogel membrane system to be simulated by Materials Studio software, to obtain a configuration file;

3) optimizing the molecular dynamics model of the hydrogel membrane system optimizing the molecular dynamics model of the hydrogel membrane system constructed in step 2) by using an energy minimization method;

4) performing a molecular dynamics simulation of the optimized model performing molecular dynamics simulations of constant number of particles, volume, and temperature (NVT), constant number of particles, pressure, and temperature (NPT), and NVT of the optimized model sequentially, to obtain a trajectory file and mean square displacement (MSD) data of a water-soluble monomer molecule in the hydrogel membrane system;

5) drawing a MSD-time (MSD-t) curve

drawing a MSD-t curve by corresponding the trajectory file and MSD data obtained from the simulation to a time; and

6) calculating a diffusion coefficient of the water-soluble monomer molecule in the hydrogel membrane system linearly fitting the MSD-t curve to calculate a slope of the fitted curve, and calculating the diffusion coefficient of the water-soluble monomer molecule in the hydrogel membrane system by an Einstein diffusion equation.

Further, step 2) is specifically:

2.1) constructing an initial three-dimensional molecular dynamics model of the hydrogel membrane material, the water-soluble monomer molecule and a water molecule through Materials Visualizer module in the Materials Studio software;

2.2) using Clean tool in the Materials Studio software to perform a preliminary optimization of the constructed initial molecular dynamics model in conformity with a chemical structure, and on this basis, using Discover module to perform energy minimization of the model to obtain the most stable molecular configuration; and

2.3) using Amorphous Cell module in the Materials Studio software to construct a cube box of the hydrogel membrane system; placing a certain number of water-soluble monomer molecules after the structure optimization according to a concentration of the water-soluble monomer required for interfacial polymerization to form a lattice model of the hydrogel membrane system; setting a system parameter including: temperature, geometric configuration number, initial density and final density.

Further, step 3) is specifically: using Geometry Optimization function of Forcite module in the Materials Studio software to perform energy minimization and NVT ensemble dynamic simulation on a successfully constructed lattice model of the hydrogel membrane system to optimize a system configuration.

Further, step 4) is specifically: using Dynamics function of the Forcite module in the Materials Studio software to sequentially perform 100 ps NVT, 100 ps NPT and 100 ps NVT dynamics simulations on a system model with the minimum energy, and obtaining a trajectory file of the water-soluble monomer molecule after equilibration.

Further, step 5) is specifically: drawing a MSD-t curve of the MSD data of the water-soluble monomer with time, where the MSD data is obtained by Formula (1):

$$M S D = \frac{1}{N} {\sum }_{1}^{N} \{ {[ r ( t ) - r ( 0 ) ]}^{2} \} \qquad ( 1 )$$

where, r(0) represents a position of the water-soluble monomer at time 0, and r(0) represents a position of the water-soluble monomer at time t.

Further, step 6) is specifically: linearly fitting the MSD-t curve; calculating a slope k of the fitted curve according to Formula (2); calculating a diffusion coefficient D according to an Einstein diffusion equation in Formula (3):

$$\begin{matrix}k = {lim}_{n \to ∞} \frac{d}{d x} \{ {[ r ( t ) - r ( 0 ) ]}^{2} \} & ( 2 ) \\ D = \frac{1}{6} k & ( 3 )\end{matrix}$$

Further, the hydrogel membrane material is one of polyparaphenylene terephthalamide, chitosan, cellulose, sodium alginate or polyvinyl alcohol.

Further, the hydrogel membrane material is preferably polyparaphenylene terephthalamide.

Further, the water-soluble monomer is one or more of piperazine, 2-methylpiperazine, 2,5-dimethylpiperazine, 4-aminomethylpiperazine, 2,5-diethylpiperazine, α-cyclodextrin, β-cyclodextrin, γ-cyclodextrin, δ-cyclodextrin, p-phenylenediamine, m-phenylenediamine, mesitylenetriamine, diaminotoluene, ethylenediamine, propanediamine, phenyldimethyldiamine, 1,3-diaminocyclohexane or 1,4-diaminocyclohexane; the water-soluble monomer has a concentration of 0.01-8.0 wt %.

Further, the water-soluble monomer is preferably piperazine, m-phenylenediamine or cyclodextrin.

The present invention has the following beneficial effects. The present invention uses molecular dynamics simulation technology to quantitatively analyze a surface microscopic characteristic and a dynamic change process of a water-soluble monomer in a hydrogel membrane system at a molecular level. The present invention predicts the diffusion performance of the water-soluble monomer in the hydrogel membrane system by a method of molecular dynamics simulation. The present invention provides a theoretical basis for exploring an influence of a gel system on interfacial polymerization and membrane separation performance, and provides data support for preparing a high-performance membrane material by using a hydrogel.

### BRIEF DESCRIPTION OF DRAWINGS

FIG. 1 is a schematic diagram of a molecular dynamics simulation of a piperazine (PIP) molecule in a hydrogel membrane system formed of polyparaphenylene terephthalamide (PPTA) and an aqueous solution under an equilibrium state.

FIG. 2 is a schematic diagram of a molecular dynamics simulation of a piperazine (PIP) molecule in a pure aqueous solution under an equilibrium state.

FIG. 3 is a mean square displacement-time (MSD-t) curve of a piperazine (PIP) molecule in a pure aqueous solution and a hydrogel membrane system formed of polyparaphenylene terephthalamide (PPTA) and an aqueous solution.

### DETAILED DESCRIPTION

To better understand the present invention, the technical solution of the present invention is described in further detail below with reference to specific implementations, but the present invention is not limited thereto.

Specific Implementation 1

This implementation scheme uses Materials Studio software to perform a molecular dynamics simulation of a diffusion property of a water-soluble monomer in a hydrogel membrane on a calculation server, including the following steps:

1) Select a hydrogel membrane material and a water-soluble monomer to be simulated.

2) Construct an initial model, specifically:

2.1) construct an initial three-dimensional molecular dynamics model of the hydrogel membrane material, a water-soluble monomer molecule and a water molecule through Materials Visualizer module in the Materials Studio software;

2.2) use Clean tool in the Materials Studio software to perform a preliminary optimization of the constructed initial molecular dynamics model in conformity with a chemical structure, and on this basis, use Discover module to perform energy minimization of the model to obtain the most stable molecular configuration, where a specific setting includes a minimization method “smart minimizer”, a steepest descent method, a conjugate gradient method and a newton method, with a convergence level being “customized” and a maximum number of iterations being 5,000; and

2.3) use Amorphous Cell module in the Materials Studio software to construct a cube box of the hydrogel membrane system; place a certain number of water-soluble monomer molecules after the structure optimization according to a concentration of the water-soluble monomer required for interfacial polymerization to form a lattice model of the hydrogel membrane system, with a periodic boundary; set a system parameter including: temperature, geometric configuration number, initial density and final density.

3) Optimize the molecular dynamics model of the hydrogel membrane system

Use Geometry Optimization function of Forcite module in the Materials Studio software to perform energy minimization and constant number of particles, volume, and temperature (NVT) ensemble dynamic simulation on a successfully constructed lattice model of the hydrogel membrane system to optimize a system configuration, where a specific setting includes a force field of condensed-phase optimized molecular potentials for atomistic simulation studies (COMPASS), an electrostatic interaction “Ewald”, a simulation temperature 298.15 K, a calculation time step 1 fs and a total simulation time 1-2 ns; all motion and coordinate parameters are selected to save a trajectory, and a simulation result is output every 5,000 steps; a maximum energy shift during simulation is 5,000 kcal/mol by default; an Andersen method is used to ensure constant system temperature; a molecular dynamics calculation is performed on a preliminary optimization result to obtain a cut-off distance of a middle-long range interaction force being 12.5 Å.

4) Perform a molecular dynamics simulation of the optimized model

Use Dynamics function of the Forcite module in the Materials Studio software to sequentially perform 100 ps NVT, 100 ps NPT and 100 ps NVT dynamics simulations on a system model with the minimum energy, and obtain a trajectory file of the water-soluble monomer molecule after equilibration.

5) Draw a MSD-t curve

Specifically: draw a MSD-t curve of the MSD data of the water-soluble monomer with time, where the MSD data is obtained by Formula (1):

$$M S D = \frac{1}{N} {\sum }_{1}^{N} \{ {[ r ( t ) - r ( 0 ) ]}^{2} \} \qquad ( 1 )$$

where, r(0) represents a position of the water-soluble monomer at time 0, and r(0) represents a position of the water-soluble monomer at time t.

6) Calculate a diffusion coefficient of the water-soluble monomer molecule in the hydrogel membrane system

Linearly fit the MSD-t curve; calculate a slope k of the fitted curve according to Formula (2);

calculate a diffusion coefficient D according to an Einstein diffusion equation in Formula (3):

$$\begin{matrix}k = {lim}_{n \to ∞} \frac{d}{d x} \{ {[ r ( t ) - r ( 0 ) ]}^{2} \} & ( 2 ) \\ D = \frac{1}{6} k & ( 3 )\end{matrix}$$

Specific Implementation 2

Take the diffusion of a piperazine (PIP) molecule in a hydrogel membrane system formed of polyparaphenylene terephthalamide (PPTA) and water as an example, this implementation specifically includes the following steps:

1) Select the PIP molecule and the PPTA.

2) Construct a molecular dynamics model of the PIP molecule in the hydrogel membrane system formed of the PPTA during interfacial polymerization, and assign a physical meaning to obtain a configuration file, specifically:

2.1) construct an initial three-dimensional molecular dynamics model of PPTA, PIP and water molecules through Materials Visualizer module in the Materials Studio software, where PPTA adopts a structure with two repeat units;

2.2) use Clean tool in the Materials Studio software to perform a preliminary optimization of the constructed initial model of each molecule in conformity with a chemical structure, and on this basis, use Discover module to perform energy minimization of each model to obtain the most stable molecular configuration, where a specific setting includes a minimization method “smart minimizer”, a steepest descent method, a conjugate gradient method and a newton method, with a convergence level being “customized” and a maximum number of iterations being 5,000; and

2.3) use Amorphous Cell module in the Materials Studio software to construct a cube box of the PIP+PPTA hydrogel membrane system with a volume of 23.26×23.26×23.26 Å³; place 320 water molecules, 2 PPTA molecules and 10 PIP molecules respectively after the structure optimization, where the system model is set to have a periodic boundary, with a temperature 298.15K, a number of geometric configurations 10, an initial density 0.6 g/cm³ and a final density 1.0 g/cm³; a concentration of the PIP in this system is calculated as 1.5 mol/L.

3) Optimize the molecular dynamics model of the PIP+PPTA hydrogel membrane system by using an energy minimization method so that the model is structurally stabilized, specifically:

use Geometry Optimization function of Forcite module in the Materials Studio software to perform energy minimization and NVT ensemble dynamic simulation on the system to optimize a system configuration, where a specific setting includes a force field of COMPASS, an electrostatic interaction “Ewald”, a simulation temperature 298.15 K, a calculation time step 1 fs and a total simulation time 1-2 ns; all motion and coordinate parameters are selected to save a trajectory, and a simulation result is output every 5,000 steps; a maximum energy shift during simulation is 5,000 kcal/mol by default; an Andersen method is used to ensure constant system temperature; a molecular dynamics calculation is performed on a preliminary optimization result to obtain a cut-off distance of a middle-long range interaction force being 12.5 Å.

4) Perform a molecular dynamics simulation and output a trajectory file and mean square displacement (MSD) data of the PIP molecule in the PPTA hydrogel membrane system, specifically:

use Dynamics function of the Forcite module in the Materials Studio software to perform 100 ps NVT and 100 ps NPT dynamics simulations on a system model with the minimum energy to equilibrate the system; then perform a 100 ps NVI dynamics simulation so that the system is finally equilibrated; save a final trajectory of the PIP molecule in the PPTA hydrogel membrane system and extract MSD data thereof.

5) Draw a MSD-t curve of the MSD data of the water-soluble monomer with time, where the MSD data is obtained by Formula: where r(0) represents a position of the PIP at time 0, and r(t) represents a position of the PIP at time t.

6) Linearly fit the MSD-t curve; calculate a slope k of the fitted curve according to Formula; calculate a diffusion coefficient D according to an Einstein diffusion equation.

A molecular dynamics simulation is performed on a PIP+PPTA hydrogel membrane system according to the above method. FIG. 1 shows a simulation of a PIP molecule in a hydrogel membrane system formed of a PPTA and an aqueous solution under an equilibrium state.

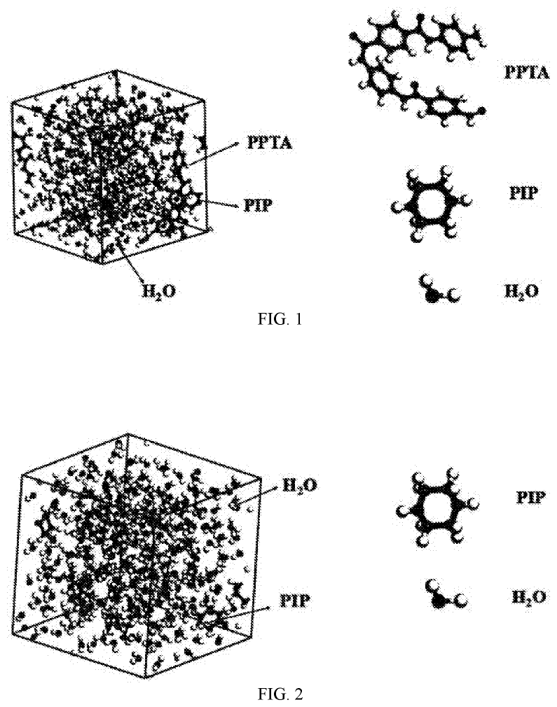

For comparison, a molecular dynamics simulation is performed on the diffusion of the PIP molecule in a pure aqueous solution system. FIG. 2 shows a schematic diagram of the PIP molecule in the pure aqueous solution under an equilibrium state.

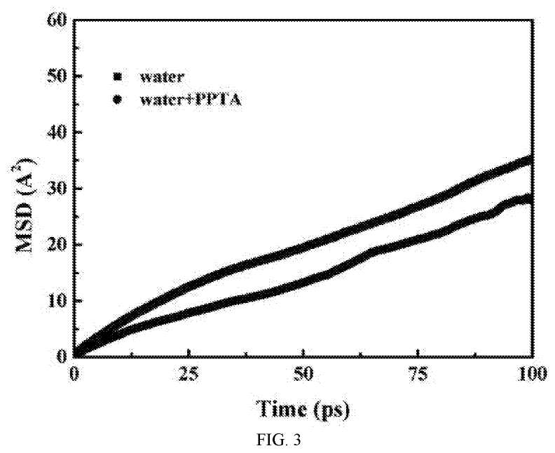

A MSD-t curve of the PIP in the two systems is obtained according to the simulations in the two systems, as shown in FIG. 3. By calculation, a diffusion coefficient of the PIP in the pure aqueous solution and the hydrogel membrane system is 0.61×10⁻⁹ m²/s and 0.45×10⁻⁹ m²/s, respectively. A calculation result analysis shows that a diffusion rate of the PIP molecule in the hydrogel membrane system formed of the PPTA and water is lower than that in the pure water system. A smaller diffusion coefficient indicates a more stable system which is the less likely for diffusion. As for interfacial polymerization for the preparation of a high-performance composite membrane, a high-molecular polymer (such as the PPTA) added to a water phase forms a hydrohydrogel membrane with water to effectively prevent the diffusion of the PIP monomer molecule to a polymerization interface. The resulting polymer membrane has a smaller thickness and a greater flux than those with the use of the pure water system. Therefore, it is possible to prepare a high-performance composite membrane by using a hydrogel membrane which hinders the diffusion of a water-soluble monomer.

The above describes the preferred implementations of the present patent in detail, but the present patent is not limited thereto. A person of ordinary skill in the art may make various changes without departing from the spirit of the present patent.

What is claimed is:

1. A simulation method for analyzing a diffusion property of a water-soluble monomer in a hydrogel membrane, comprising the following steps:
1) selecting a hydrogel membrane material and a water-soluble monomer to be simulated;
2) constructing an initial model
constructing a molecular dynamics model of a hydrogel membrane system to be simulated by Materials Studio software, to obtain a configuration file;
3) optimizing the molecular dynamics model of the hydrogel membrane system
optimizing the molecular dynamics model of the hydrogel membrane system constructed in step 2) by using an energy minimization method;
4) performing a molecular dynamics simulation of the optimized model
performing molecular dynamics simulations of constant number of particles, volume, and temperature (NVT), constant number of particles, pressure, and temperature (NPT), and NVT of the optimized model sequentially, to obtain a trajectory file and mean square displacement (MSD) data of a water-soluble monomer molecule in the hydrogel membrane system;
5) drawing a MSD-t curve
drawing a MSD-t curve by corresponding the trajectory file and MSD data obtained from the simulation to a time; and
6) calculating a diffusion coefficient of the water-soluble monomer molecule in the hydrogel membrane system
linearly fitting the MSD-t curve to calculate a slope of the fitted curve, and calculating the diffusion coefficient of the water-soluble monomer molecule in the hydrogel membrane system by an Einstein diffusion equation.

2. The simulation method for analyzing a diffusion property of a water-soluble monomer in a hydrogel membrane according to claim 1, wherein step 2) is specifically:
2.1) constructing an initial three-dimensional molecular dynamics model of the hydrogel membrane material, the water-soluble monomer molecule and a water molecule through Materials Visualizer module in the Materials Studio software;
2.2) using Clean tool in the Materials Studio software to perform a preliminary optimization of the constructed initial molecular dynamics model in conformity with a chemical structure, and on this basis, using Discover module to perform energy minimization of the model to obtain the most stable molecular configuration; and
2.3) using Amorphous Cell module in the Materials Studio software to construct a cube box of the hydrogel membrane system; placing a certain number of water-soluble monomer molecules after the structure optimization according to a concentration of the water-soluble monomer required for interfacial polymerization to form a lattice model of the hydrogel membrane system; setting a system parameter comprising: temperature, geometric configuration number, initial density and final density.

3. The simulation method for analyzing a diffusion property of a water-soluble monomer in a hydrogel membrane according to claim 1, wherein step 3) is specifically:
using Geometry Optimization function of Forcite module in the Materials Studio software to perform energy minimization and NVT ensemble dynamic simulation on a successfully constructed lattice model of the hydrogel membrane system to optimize a system configuration.

4. The simulation method for analyzing a diffusion property of a water-soluble monomer in a hydrogel membrane according to claim 1, wherein step 4) is specifically:
using Dynamics function of the Forcite module in the Materials Studio software to sequentially perform 100 ps NVT, 100 ps NPT and 100 ps NVT dynamics simulations on a system model with the minimum energy, and obtaining a trajectory file of the water-soluble monomer molecule after equilibration.

5. The simulation method for analyzing a diffusion property of a water-soluble monomer in a hydrogel membrane according to claim 1, wherein step 5) is specifically:
drawing a MSD-t curve of the MSD data of the water-soluble monomer with time, wherein the MSD data is obtained by Formula (1):
$$M S D = \frac{1}{N} {\sum }_{1}^{N} \{ {[ r ( t ) - r ( 0 ) ]}^{2} \} \qquad ( 1 )$$
wherein, r(0) represents a position of the water-soluble monomer at time 0, and r(0) represents a position of the water-soluble monomer at time t.

6. The simulation method for analyzing a diffusion property of a water-soluble monomer in a hydrogel membrane according to claim 1, wherein step 6) is specifically: linearly fitting the MSD-t curve; calculating a slope k of the fitted curve according to Formula (2); calculating a diffusion coefficient D according to an Einstein diffusion equation in Formula (3):
$$\begin{matrix}k = {lim}_{n \to ∞} \frac{d}{d x} \{ {[ r ( t ) - r ( 0 ) ]}^{2} \} & ( 2 ) \\ D = \frac{1}{6} k . & ( 3 )\end{matrix}$$

7. The simulation method for analyzing a diffusion property of a water-soluble monomer in a hydrogel membrane according to claim 1, wherein the hydrogel membrane material is one of polyparaphenylene terephthalamide, chitosan, cellulose, sodium alginate or polyvinyl alcohol.

8. The simulation method for analyzing a diffusion property of a water-soluble monomer in a hydrogel membrane according to claim 1, wherein the hydrogel membrane material is preferably polyparaphenylene terephthalamide.

9. The simulation method for analyzing a diffusion property of a water-soluble monomer in a hydrogel membrane according to claim 1, wherein the water-soluble monomer is one or more of piperazine, 2-methylpiperazine, 2,5-dimethylpiperazine, 4-aminomethylpiperazine, 2,5-diethylpiperazine, α-cyclodextrin, β-cyclodextrin, γ-cyclodextrin, δ-cyclodextrin, p-phenylenediamine, m-phenylenediamine, mesitylenetriamine, diaminotoluene, ethylenediamine, propanediamine, phenyldimethyldiamine, 1,3-diaminocyclohexane or 1,4-diaminocyclohexane; the water-soluble monomer has a concentration of 0.01-8.0 wt %.

10. The simulation method for analyzing a diffusion property of a water-soluble monomer in a hydrogel membrane according to claim 1, wherein the water-soluble monomer is preferably piperazine, m-phenylenediamine or cyclodextrin.
